(19) United States
(12) Patent Application Publication
(10) Pub. No.: US 2022/0118118 A1
(43) Pub. Date: Apr. 21, 2022

(54) BENZAZEPIN-L,7-DIOL-DERIVED RADIOLABELED LIGANDS WITH HIGH IN VIVO NMDA SPECIFICITY

(71) Applicant: ETH ZURICH, Zurich (CH)

(72) Inventors: Simon M. AMETAMEY, Zurich (CH); Ahmed HAIDER, Zug (CH); Hazem AHMED, Zurich (CH)

(21) Appl. No.: 17/294,187

(22) PCT Filed: Nov. 14, 2019
(86) PCT No.: PCT/EP2019/081281

§ 371 Date: May 14, 2021

(30) Foreign Application Priority Data
Nov. 15, 2018 (EP) 18206483.2

(51) Int. Cl. A61K 51/04; A61K 49/00

(52) U.S. Cl. CPC A61K 51/0468; A61K 49/0004

(57) ABSTRACT

The present invention is directed to benzazepin-1,7-diol-derived compounds (I) for use in the diagnosis of NMDA (N-methyl-D-aspartate) receptor-associated diseases or disorders by positron emission tomography (PET), single-photon emission computed tomography (SPECT), liquid based-scintillation- and/or autoradiography-based assays. The invention also relates to a method for the diagnosis of NMDA receptor-associated diseases or disorders by administering to a patient or a sample of a patient in need of such diagnosis a compound of the invention in an amount effective for PET imaging, SPECT imaging, liquid based-scintillation- and/or autoradiography-based assays of NMDA receptors, recording at least one PET or SPECT scan, liquid based-scintillation or autoradiography result, and diagnosing an NMDA receptor-associated disease or disorder from an abnormal NMDA receptor expression pattern on the PET or SPECT scan, in the liquid based-scintillation or autoradiography result. The present invention also provides a method for evaluating a putative NMDA-receptor antagonist in a liquid scintigraphy detection assay or an autoradiography assay using the compounds of the present invention.

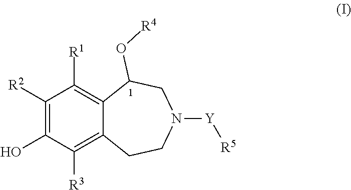

### RELATED APPLICATIONS

This application is a National Stage of PCT/EP2019/081281, filed 14 Nov. 2019, titled BENZAZEPIN-L,7-DIOL-DERIVED RADIOLABELED LIGANDS WITH HIGH IN VIVO NMDA SPECIFICITY, published as International Patent Application Publication No. WO 2020/099537 A1, which claims the benefit of and priority to European Patent Application No. 18206483.2, filed on 15 Nov. 2018, both of which are incorporated herein by reference in their entirety for all purposes.

### FIELD OF THE INVENTION

The present invention is directed to benzazepin-1,7-diol-derived compounds for use in the diagnosis of NMDA (N-methyl-D-aspartate) receptor-associated diseases or disorders by positron emission tomography (PET), single-photon emission computed tomography (SPECT), liquid based scintillation- and/or autoradiography-based assays. The invention also relates to a method for the diagnosis of NMDA receptor-associated diseases or disorders by administering to a patient or a sample of a patient in need of such diagnosis a compound of the invention in an amount effective for PET imaging, SPECT imaging, liquid based-scintillation- and/or autoradiography-based assays of NMDA receptors, recording at least one PET or SPECT scan, liquid based-scintillation or autoradiography result, and diagnosing an NMDA receptor-associated disease or disorder from an abnormal NMDA receptor expression pattern on the PET or SPECT scan, in the liquid based-scintillation or autoradiography result. The present invention also provides a method for evaluating a putative NMDA-receptor antagonist in a liquid scintigraphy detection assay or an autoradiography assay using the compounds of the present invention.

### BACKGROUND OF THE INVENTION

The functional complexity of the NMDA receptor family and the diversity of ligand binding sites discovered in recent years offer a wide variety of options for modulating neuronal activity. However, recent experiences including a few disappointing clinical trials have shown that this complexity renders NMDA receptors challenging targets in drug development (Monaghan et al., Neurochem. Int. 2012, 61, 581-592; Curr Opin Pharmacol 2015, 20, 14-23). A functional NMDA receptor consists of four subunits, involving two or three of the seven homologous gene products GluN1, GluN2A-D, GluN3A and 3B. The subunit composition is highly adaptive and depends on the macro- and microscopic location of the receptor, the developmental age, neuronal function and activity (Paoletti et al., Nat. Rev. Neurosci. 2013, 14, 383-400). Due to the diverse and sometimes opposite functions of the individual receptor heterotetramers, subtype-selective compounds became of considerable interest in drug development.

However, despite great efforts in drug research towards GluN1/GluN2B-selective NTD (N-terminal domain) ligands, results from clinical trials were disappointing and did not meet the expectations from basic and preclinical research (Ikonomidou and Turski, Lancet Neurol. 2002, 1, 383-386). Comparing results from in vitro and in vivo experiments, the discrepancies between experimental Ki values (affinity to GluN1/GluN2B receptors) and concentrations or doses that were required to induce a particular pharmacodynamic or pharmacological response are striking in many cases. Several in vitro experiments indicated binding affinities and pharmacodynamic effects of GluN1/GluN2B-selective NTD modulators in the low nanomolar concentration range while other work reported significant binding and receptor-related effects only in the high nanomolar or low micromolar range (Schepmann et al., J. Pharm. Biomed. Anal. 2010, 53, 603-608). Several of these studies showed a high and a low affinity interaction with native (mixed tetraheteromeric) receptors, independent of the absolute values, in agreement with the reported high and low affinity binding to recombinant GluN1/GluN2B and GluN1/GluN2A, respectively. Considering the high brain uptake of eliprodil with brain/plasma ratios of about 20 (Garrigou-Cadenne et al., J Pharmacokinet Biopharm 1995, 23, 147-161) and assuming low nanomolar binding affinity (Tewes et al., ChemMedChem 2010, 5, 687-695), effective doses of eliprodil in preclinical in vivo studies were magnitudes higher than what would be expected sufficient to occupy a high portion of the GluN1/GluN2B NTD binding sites (Toulmond et al., Brain Res. 1993, 620, 32-41). Furthermore, while regional expression levels of the individual subunits of the NMDA receptors are known (Laurie et al., Brain Res Mol Brain Res 1997, 51, 23-32), the in vivo regional binding pattern of the GluN1/GluN2B NTD-selective drugs remains elusive. The contradictive findings encouraged academic and industrial research teams to develop modulators with improved pharmacodynamic properties, in particular regarding affinity and selectivity (Strong et al., Expert Opin. Ther. Pat. 2014, 24, 1349-1366; Tewes et al., ChemMedChem 2010, 5, 687-695). In addition to an improved selectivity pattern of the modulators, methods are required to optimize dosage schemes towards optimal receptor subtype occupancy at minimal binding to off-targets including alternative NMDA receptor subtypes.

Tewes et al. (ChemMedChem 2010, 5, 687-695) describes the synthesis and biological evaluation of 3-benzazepins as NR2B-selective NMDA receptor antagonists. In competition assays using tritium-labeled Ifenprodil as radioligand, affinity towards NR2B-containing NMDA receptors in membrane homogenates of cells stably expressing recombinant human NR1a/NR2B receptors (dexamethasone-induced, ketamine (NMDA antagonist) stabilized for avoiding cell death) was demonstrated for a number of 3-benzazepins. For investigating the selectivity of these 3-benzazepin-derived NR2B ligands, the compounds were tested against the phencyclidine (PCP) binding site of the NMDA receptor and both a (sigma) receptor subtypes (σ₁ and σ₂) in receptor binding studies. The benzazepines did not show significant interactions with the PCP binding site while showing high selectivity for the polyamine binding site of the NMDA receptor. The affinities towards the σ₂-receptor were also generally low. With regard to the σ₁-receptor, it was shown that structural changes in the 3-benzazepines can shift the receptor profile from an NR2B-selective ligand to a σ₁-selective ligand. Even the development of GluN2B-antagonists yielded promising preclinical results as well as highly affine and selective NR2B NMDA receptor antagonists with potential for therapeutic use, clinical trials did not establish sufficient therapeutic benefit for medical use to date (Addy et al., J Clin Pharmacol, 49, 856-864, 2009).

Positron emission tomography (PET) is a nuclear medicine, functional imaging technique that produces a three-dimensional image of functional processes in the body. The system detects pairs of gamma rays emitted indirectly by a positron-emitting radionuclide (tracer), which is introduced into the body on a biologically active molecule. Three-dimensional images of tracer concentrations within the body are then constructed by computer analysis. In modern PET-CT scanners, three dimensional imaging is often accomplished with the aid of a CT X-ray scan performed on the patient during the same session, integrated in the same machine. PET and singlephoton emission computed tomography (SPECT) are valuable techniques for research and pharmacodynamic pre-clinical studies of potential medicaments and they are used regularly for diagnosing certain diseases and disorders.

Because the radioactive tracer forms part of a biologically active and target-specific molecule, the so-called PET or SPECT ligand, the PET or SPECT technique images the target-specific distribution of the tracer in healthy and diseased live tissue. Depending on the target specificity of the PET or SPECT ligand an abnormal biodistribution of targets can be indicative of diseases and disorders. For example, PET or SPECT imaging is useful for diagnosing tumors and sites of metastases (oncology), for imaging neurodegenerative disease such as Alzheimer's disease, for localizing a seizure focus, for imaging psychiatric disorders such as schizophrenia, substance abuse, mood disorders (neuroimaging), for imaging artherosclerosis and vascular diseases (cardiology and neurology), and for imaging bacterial infections.

It is a common problem of PET ligands that there is slow or insufficient biodistribution or even no penetration of the ligands through certain tissues, e.g. the blood brain barrier. Furthermore, PET ligand specificity for the aimed target is regularly compromised by unspecific binding of the ligand to non-targeted proteins such as serum albumin. All these shortcomings lead to low quality PET images which lack a proper signal to noise ratio or display artifacts.

There are many PET ligands for neuroimaging, e.g. ¹¹C- and ¹⁸F-labelled compounds such as Raclopride, Fallypride, Desmethoxyfallypride for dopamine D2/D3 receptors, McN 5652 and DASB for serotonin transporters, Mefway for serotonin 5HT1A receptors, Nifene for nicotinic acetylcholine receptors and a number of amyloid protein-specific PET ligands. However, at present there are no NMDA-specific PET ligands with an in vivo specificity that is sufficiently high, i.e. much higher than 30%, to correctly reflect the NMDA receptor biodistribution in patients with NMDA receptor-associated diseases or disorders.

US 2017/0224852 A1 discloses chemically stable PET ligands that bind selectively to the NMDA receptor, in particular to the GluN2B subunit. These PET ligands bind with good affinity in all major brain regions and accumulate heterogeneously in different regions of a rat brain. The PET ligands reported in US 2017/0224852 A1 are suitable for non-invasively imaging the density of GluN2B(C,D)-containing NMDA receptors in the mammalian brain and for assessing the degree of receptor occupancy by GluN1/GluN2B NTD modulators.

In view of the above, it is the objective of the present invention to provide further and/or optionally improved radiolabeled ligands, e.g. PET or SPECT ligands, with high NMDA receptor affinity and high NMDA receptor selectivity that are suitable for use in the diagnosis of NMDA receptor-associated diseases or disorders, e.g. by positron emission tomography (PET), single-photon emission computed tomography (SPECT), liquid based-scintillation and autoradiography assays with good biodistribution, high signal to noise ratio and little artifact generation.

In a first aspect, the objective of the present invention is solved by the following compounds of formula (I):

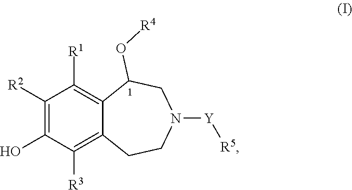

wherein
at least one atom of formula (I) is a radiolabeled atom, optionally a radiolabeled atom selected from the group consisting of ³H—; ¹¹C—; ¹⁴C—; ¹⁸F—; ¹³N—; ¹⁵O—; ¹²³I—; ¹²⁴I—; ¹²⁵I—; ¹³¹I—; and positron emitting radiometals, optionally ⁶⁴Cu and ⁶⁸Ga, suitable for detection in a method selected from the group consisting of positron emission tomography (PET), single-photon emission computed tomography (SPECT), liquid based-scintillation counting assays, and autoradiography;
one of R¹, R² and R³ is independently selected from the group consisting of hydrogen, deuterium, tritium, fluorine, chlorine, bromine, iodine, —OH, —CN, —NO₂, —NH₂, —SH₂, —(C₁-C₄)alkyl, fluorinated —(C₁-C₄)alkyl, optionally —CH₂F, —CD₂F, —CT₂F, FCH₂CH₂—, FCH₂CH₂CH₂—, —O(C₁-C₄)alkyl, and fluorinated —O(C₁-C₄)alkyl, optionally —OCH₂F, —OCD₂F, FCH₂CH₂O—, and FCH₂CH₂CH₂O—,
and the other of R¹ to R³ are hydrogen or fluorine, wherein T is ³H—;
R⁴ is selected from the group consisting of
hydrogen, —(C₁-C₄)alkyl, fluorinated —(C₁-C₄)alkyl, chlorinated —(C₁-C₄)alkyl, brominated —(C₁-C₄)alkyl, deuterated —(C₁-C₄)alkyl and tritiated —(C₁-C₄)alkyl, optionally —CH₂F, —CD₂F, —CT₂F, FCH₂CH₂—, FCH₂CH₂CH₂—, FCD₂CD₂-, FCD₂CD₂CD₂-, FCT₂CT₂-, FCT₂CT₂CT₂-, —CH₂Cl, —CD₂Cl, —CT₂I, ClCH₂CH₂—, ClCH₂CH₂CH₂—, ClCD₂CD₂-, ClCD₂CD₂CD₂-, ClCT₂CT₂-, ClCT₂CT₂CT₂-, —CH₂I, —CD₂I, —CT₂I, ICH₂CH₂—, ICH₂CH₂CH₂—, ICD₂CD₂-, ICD₂CD₂CD₂-, ICT₂CT₂-, and ICT₂CT₂CT₂-, and optionally R¹ to R³ are hydrogen, fluorine, chlorine, bromine or iodine.
Y is selected from the group consisting of
(C₁-C₆)alkyl, optionally C₅-alkyl or C₄-alkyl,
(C₁-C₆)alkyl substituted with at least one deuterium, tritium, ¹⁸F, ¹¹F, chlorine, bromine, iodine and/or OH, optionally CH₂—CH₂—FCH—CH₂N—, CH₂—FCH—CH₂CH₂N—, CH₂CH₂—ICH—CH₂N—, CH₂—ICH—CH₂CH₂N—, CH₂CH₂—CD₂-CH₂N—, CH₂—CD₂-CD₂CH₂N—, CH₂CH₂—CT₂-CH₂N—, CH₂—CT₂-CT₂CH₂N—, or CH₂—CHOH—CH₂—CH₂N—,
(C₁-C₆)alkoxyalkyl, (C₁-C₆)polyethyleneglycoyl, optionally —(CH₂)₂—O—(CH₂)₂—R⁵,
—(CH₂)₃—O—R⁵, —(CH₂)₄—O—R⁵, and
(C₁-C₆)heteroalkyl, optionally —(CH₂)₃—X—R⁵ and —(CH₂)₄—X—R⁵, wherein X is sulfur or SO₂,

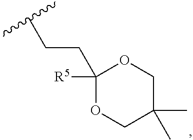

—(CH₂)₃—CO—R⁵, —(CH₂)₂—CO—N(CH₃)—CH₂—R⁵, and —CO—(CH₂)₃—R⁵;
R⁵ is selected from the group consisting of
substituted or non-substituted (C₅-C₆)aryl, substituted or non-substituted (C₅-C₆)heteroaryl, optionally substituted or non-substituted phenyl or pyridyl,
optionally

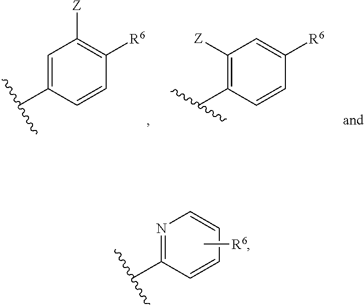

wherein Z is selected from the group consisting of hydrogen, tritium, fluorine, chlorine, bromide, iodine, cyano and nitrile, wherein R⁶ is selected from the group consisting of hydrogen, deuterium, tritium, fluorine, chlorine, bromine, iodine, —(C₁-C₆)alkyl, fluorinated —(C₁-C₆)alkyl, optionally —CH₂F, —CD₂F, FCH₂CH₂—, FCH₂CH₂—, and FCH₂CH₂CH₂—; and/or
YR⁵ is selected from the group consisting of:

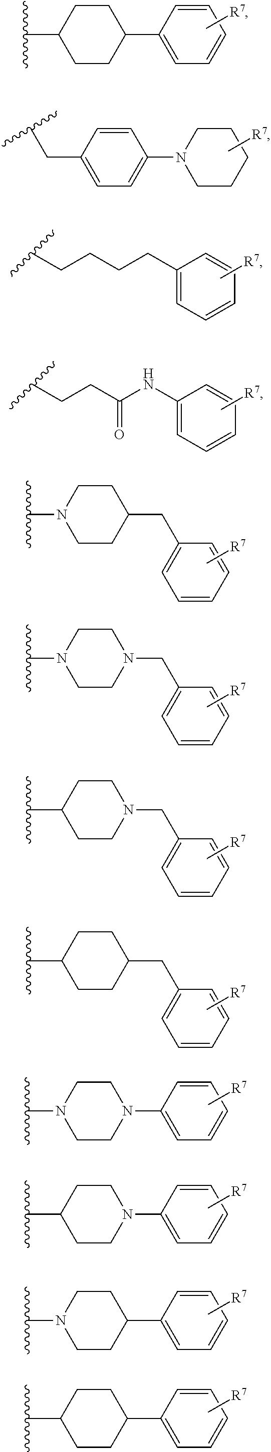

wherein R⁷ is one or more hydrogen, deuterium, tritium, fluorine, chlorine, bromine, iodine or (C₁-C₇)alkyl substituted with at least one deuterium, tritium, fluorine, chlorine, bromine, iodine or OH;
   and pharmaceutically acceptable salts or solvates thereof, for use in the diagnosis of NMDA receptor-associated diseases or disorders by positron emission tomography (PET), single-photon emission computed tomography (SPECT), liquid based-scintillation counting assays, or autoradiography.

In the context of the present invention it is understood that antecedent terms such as “alkyl” are to be interpreted as encompassing linear or branched, substituted or non-substituted alkyl residues. The scope of the term “linear or branched, substituted or non-substituted alkyl” encompasses linear or branched, substituted or non-substituted alkyl residues. For example, the term “(C₁₋₄)alkyl” indicates the group of compounds having 1 to 4 carbons that is linear or branched, substituted or non-substituted.

Alkoxyalkyl groups as used herein shall be understood to mean any linear or branched, substituted or non-substituted alkyl chain comprising an oxygen atom either as an ether motif, i.e. an oxygen bound by two carbons, as an oxygen bound to any other chemical atom than carbon, e.g. hydroxyl group, or an oxygen anion.

The term heteroatom as used herein shall be understood to mean atoms other than carbon and hydrogen such as and optionally O, N, S, P, F, Cl, Br and I.

Heteroalkyl residues are carbon chains in which one or more carbon atoms can be optionally replaced by heteroatoms, optionally by O, N, S, P, F, Cl, Br or I. If N is not substituted it is NH. The heteroatoms may replace either terminal or internal carbon atoms within a linear or branched carbon chain. Such groups can be substituted as herein described by groups such as oxo to result in definitions such as but not limited to alkoxycarbonyl, acryl, amido and thioxo.

The term aryl as used herein shall be understood to mean an aromatic carbocycle or heteroaryl as defined herein. Each aryl or heteroaryl unless otherwise specified includes its partially or fully hydrogenated derivative. For example, quinolinyl may include decahydroquinolinyl and tetrahydroquinolinyl; naphthyl may include its hydrogenated derivatives such as tetrahydronaphthyl. Other partially or fully hydrogenated derivatives of the aryl and heteroaryl compounds described herein will be apparent to one of ordinary skill in the art. Naturally, the term encompasses aralkyl and alkylaryl, both of which are further embodiments for practicing the compounds of the present invention. For example, the term aryl encompasses phenyl, indanyl, indenyl, dihydronaphthyl, tetrahydronaphthyl, naphthyl and decahydronaphthyl.

The term heteroaryl shall be understood to mean an aromatic C₃-C₂₀, optionally 5-8 membered monocyclic or optionally 8-12 membered bicyclic ring containing 1-4 heteroatoms such as N, O and S. Exemplary heteroaryls comprise aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazole[3,4-b]pyridinyl, tubercidinyl, oxazo[4,5-b]pyridinyl, and imidazo[4,5-b]pyridinyl. Terms which are analogues of the above cyclic moieties such as aryloxy or heteroaryl amine shall be understood to mean an aryl, heteroaryl, heterocycle as defined above attached to its respective group.

As used herein, the terms nitrogen and sulphur include any oxidized form of nitrogen and sulphur and the quaternized form of any basic nitrogen as long as the resulting compound is chemically stable. For example, an —S—C₁₋₆ alkyl radical shall be understood to include —S(O)—C₁₋₆ alkyl and —S(O)₂—C₁₋₆alkyl.

The term polyethyleneglycol as used herein refers to a chain of substituted or nonsubstituted ethylene oxide monomers.

As used herein and in the context of all embodiments, the term “comprising” optionally also includes that no further components may be present, i.e. includes the term “consisting of”.

It was found that the compounds of the present invention bind selectively to the NMDA receptor, in particular to the GluN2B subunit, with an unexpected high binding affinity and selectivity over the 61 receptor (see, e.g., representative Example 5 below).

Without wishing to be bound by theory, it is believed that the hydroxyl group on the aromatic ring of the bicyclic structure of the compounds (in ortho position to R² and R³) for use according to the present invention is responsible for the improved binding affinities. A direct comparison of the free hydroxyl group ortho to R² and R³ vs. the corresponding methyl ethers demonstrated an unexpected and significant improvement of
(i) autoradiography results (see FIGS. 1, 2 and 3),
(ii) the binding affinity for the GluN2B subunit of the NMDA receptor and selectivity over the σ1 receptor (see Table 1 below), and
(iii) the in vivo uptake (reflected in the time activity curves of Example 4 and FIGS. 9, 10 and 11).

In summary, the compounds for use according to the present invention unexpectedly but clearly outperform the compounds of the state of the art.

Preferably, the compounds of the invention have an affinity to the NMDA receptor in the nanomolar range, optionally less than 100 nM. Assays for assessing NMDA receptor affinities are common general knowledge in the field and can be found, for example, in Tewes et al. (ChemMedChem 2010, 5, 687-695) and in the Examples below.

The inventive PET ligands provide high-quality nuclear medicine images, e.g., in short scan times within, e.g., 5-90 min and allow quantitative analysis of the ligand in the blood. Uptake of the present ligands into brain allows for non-invasive imaging of the density of NMDA receptors and enables to assess the degree of receptor occupancy by GluN1/GluN2B NTD modulators. The present PET ligands bind with high affinity in substantially all major GluN2B-rich brain regions in autoradiographic studies (see FIGS. 5, 6 and 7). No radioactive metabolites could be detected in the brain homogenate of rats, indicating metabolic stability of the present PET ligands (see FIG. 8).

The radioligands of the present invention exhibit high specificity for GluN2B-carrying NMDA receptors in PET experiments which demonstrated a heterogeneous accumulation of the PET ligands in different regions of the rat brain which was specifically reduced under blocking conditions by CP-101,606 (see FIGS. 10 and 11).

In conclusion, the present ligands represent improved compounds for non-invasively imaging the density of GluN2B-containing NMDA receptors in the mammalian brain and for assessing the degree of receptor occupancy by GluN1/GluN2B NTD modulators. The improved selectivity together with the improved brain accumulation make the compounds of the present invention excellent candidates for diagnostic applications relating to the ECS in mammals.

Additionally, when for example labeled with tritium and ¹⁴C-labeled, the compounds for use in the present invention can be used, e.g., for liquid based-scintillation assays such as in in vitro binding experiments, in vitro/ex vivo autoradiographies and ex vivo receptor occupancy experiments of GluN2B-antagonists, e.g. antagonists that are in development (see method aspects below). Notably, advantages of ³H- and ¹⁴C-labeled probes include, e.g., the ease of radioactivity handling (e.g. less expenses for laboratory facility and higher safety compared to positron-emitters) and the long shelf-life of the product given the long physical half-lives of these radioisotopes, which ultimately allows high throughput screening and several experiments at different time points with only one batch of production. The commercially available tritiated radioligand currently used in GluN2B-targeted drug development is non-selective ³H-ifenprodil. For example, and in contrast to ³H-ifenprodil, the compounds for use in the present invention are highly selective over sigma1 receptors and therefore provide an improved tool to evaluate NMDA targeting ligands.

In another embodiment, the PET or SPECT ligand for use in the present invention is one, wherein the at least one radiolabeled atom is a ¹¹C-atom, ¹⁸F-atom, ¹²³I-atom, ¹²⁴I-atom, ¹²¹I-atom, or ¹³¹I-atom.

The radiolabeled atom can be introduced by radiosynthetic means known in the art (see FIGS. 1 to 4 and Examples 2 and 7 below). Typically, the radiosynthesis of the present PET ligands can be accomplished within, e.g., 2.5 hours after end of bombardment and yields in, e.g., about 1 GBq at the end of the synthesis.

In a further embodiment the compound for use in the invention is one, wherein at least one of R⁴ or R⁵ comprise a ¹¹C-atom, ¹⁸F-atom, ²³I-atom, ¹²⁴I-atom, ¹²⁵I-atom, or ¹³¹I-atom, optionally R⁴ is —OCH₂CH₂-¹⁸F, —OCH₂CH₂CH₂-¹⁸F, OCH₂CH₂-¹²³I, or —OCH₂CH₂CH₂-¹²³I, and/or R⁶ is —¹⁸F or ¹²³I.

In a further embodiment one of R¹, R² and R³ is independently selected from the group consisting of —H, -D, -T, —CH₃, —CH₂F, —CD₂F, FCH₂CH₂—, FCH₂CH₂CH₂—, —OCH₃, —OCH₂F, —OCD₂F, FCH₂CH₂O— and FCH₂CH₂CH₂O—, and the other of R¹ to R⁴ are hydrogen or fluorine.

In a further embodiment, R¹ is selected from the group consisting of —CH₃, —CH₂F, —CD₂F, FCH₂CH₂—, FCH₂CH₂CH₂—, —OCH₃, —OCH₂F, —OCD₂F, FCH₂CH₂O— and FCH₂CH₂CH₂O—, and R², R³ and R⁴ are hydrogen or fluorine.

In a further embodiment, R⁴ is selected from the group consisting of hydrogen, —CH₂F, —CD₂F, FCH₂CH₂—, and FCH₂CH₂CH₂—.

In a further embodiment, Y is selected from the group consisting of —(CH₂)ᵢ—R⁵ and —(CH₂)ₑ—O—(CH₂)_{f}—R⁵, wherein i is an integer from 2 to 6, and e and f are independently selected from 1, 2 or 3.

In a further embodiment, R⁵ is selected from the group consisting of phenyl,

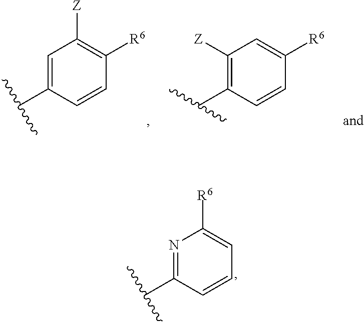

wherein Z is selected from the group consisting of hydrogen, deuterium, tritium, fluorine, chlorine, bromine, iodine, cyano and nitrile, and wherein R⁶ is selected from the group consisting of hydrogen, deuterium, tritium, fluorine, chlorine, bromine, iodine, cyano, —CH₂F, —CD₂F, FCH₂CH₂—, FCH₂CH₂—, and FCH₂CH₂CH₂—.

In a further embodiment the compound for use in the invention is one wherein R⁶ is selected from the group consisting of hydrogen, tritium, fluorine and iodine.

In a further embodiment the compound for use in the invention is one wherein R⁶ is tritium.

In another embodiment the compound for use in the invention is one, wherein the compound is R-configured at carbon 1.

In another embodiment the compound for use in the invention is one wherein the compound is S-configured at carbon 1.

In a further embodiment the compound for use in the invention is one wherein
R¹ is selected from the group consisting of —OCH₃, —OCH₂F, —OCD₂F, FCH₂CH₂O— and FCH₂CH₂CH₂O—, and R², R³ and R⁴ are hydrogen,
R⁴ is hydrogen,
Y is —(CH₂)₄—, and
R⁵ is substituted or unsubstituted phenyl.

In a further embodiment the compound for use in the invention is one, wherein
R⁴ is selected from the group consisting of hydrogen, —CH₂F, —CD₂F, FCH₂CH₂—, and FCH₂CH₂CH₂—,
Y is —(CH₂)₄— or —(CH₂)₂—O—(CH₂)₂—, and
R⁵ is selected from the group consisting of phenyl,

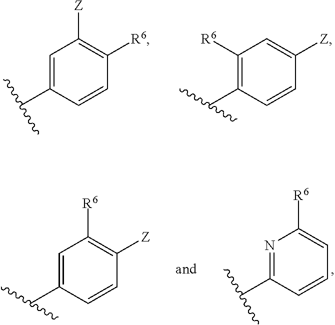

wherein Z is hydrogen, tritium or nitrile and wherein R⁶ is selected from the group consisting of fluorine, iodine, —CH₂F, —CD₂F, FCH₂—, FCH₂CH₂—, and FCH₂CH₂CH₂—.

In a further embodiment the compound for use in the invention is one wherein
R⁴ is hydrogen,
Y is (CH₂)₄, and
R⁵ is substituted or un-substituted phenyl.

In another embodiment the compound for use in the invention is selected from the group consisting of

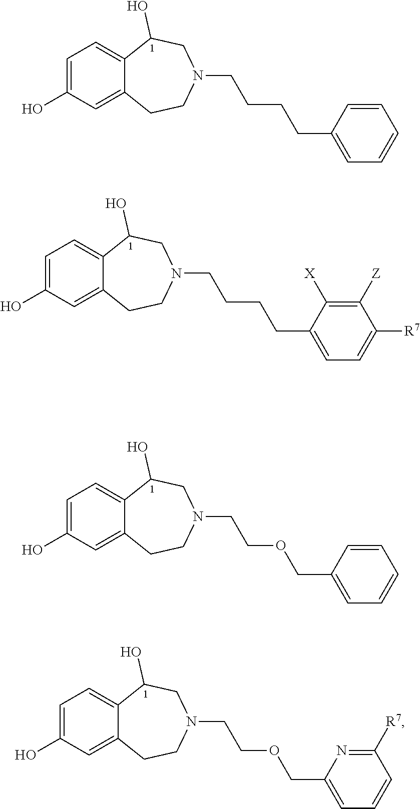

wherein
R⁷ is selected from the group consisting of fluorine, chlorine, bromine, iodine, deuterium, tritium, —CH₂F, —CD₂F, FCH₂CH₂—, FCH₂CH₂—, and FCH₂CH₂CH₂—; and
X and Z are independently selected from the group consisting of hydrogen, deuterium, tritium, fluorine, iodine and nitrile.

In a further embodiment, the compound for use in the invention is selected from the group consisting of

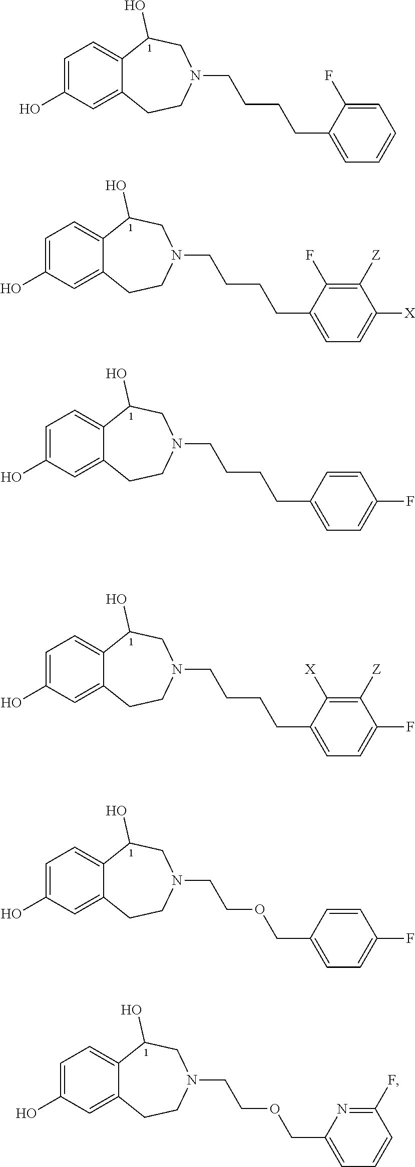

wherein
X and Z are independently selected from the group consisting of hydrogen, deuterium, tritium, fluorine, iodine and nitrile; and
the fluorine is ¹⁹F if X and/or Z are tritium and the fluorine is ¹⁸F if X and/or Z are not tritium.

The present invention includes pharmaceutically acceptable salts or solvates of the compounds of formula I. A “pharmaceutically acceptable salt or solvate” refers to any pharmaceutically acceptable salt or solvate which, upon administration to a patient, is capable of providing (directly or indirectly) a compound of the invention, or a pharmacologically active metabolite or pharmacologically active residue thereof. A pharmacologically active metabolite shall be understood to mean any compound of the invention capable of being metabolized enzymatically or chemically.

Pharmaceutically acceptable salts include those derived from pharmaceutically acceptable inorganic and organic acids and bases. Examples of suitable acids include hydrochloric, hydrobromic, sulphuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g. magnesium), ammonium and N—(C₁-C₄alkyl)₄⁺ salts.

In addition, the scope of the invention also encompasses prodrugs of compounds of formula I. Prodrugs include those compounds that, upon simple chemical transformation within a body of a patient, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

In a further aspect, the present invention is directed to a method for the diagnosis of NMDA-receptor-associated diseases or disorders comprising the following steps:

administering to a patient or a sample of a patient in need of such diagnosis a compound for use in the invention in an amount effective for PET imaging, SPECT imaging, liquid based-scintillation- and/or autoradiography-based assays of NMDA receptors,

recording at least one PET scan, SPECT scan, liquid based-scintillation or autoradiography result, optionally by ex vivo analysis, and

diagnosing an NMDA-receptor-associated disease or disorder from an abnormal NMDA receptor expression pattern on the PET scan, SPECT scan, in the liquid based-scintillation or autoradiography result.

The term patient, as used in the above context, includes human and animal patients, optionally mammals.

The term a sample of a patient, as used in the above context, is meant to include any tissue, e.g. biopsy, body liquid, e.g. blood, serum, cerebral or cerebrospinal fluid, that encompasses or is likely to encompass NMDA receptors.

Optionally, liquid based-scintillation-based assays and autoradiography assays are used for diagnosing animals, e.g. animal diseases, e.g. by ex vivo analysis of the sample, in all method aspects of the present invention.

The term liquid based-scintillation, as used herein, includes the measurement of, e.g., a sample by using the technique of mixing the sample with a liquid scintillator in order to enable counting of the radiation, e.g. the resultant photon emissions. For example, the purpose is to enhance the signal of the sample by direct contact of the radioisotope with the scintillator.

Scintillation-based or autoradiography-based assays are meant to include any scintillation assay or autoradiography assay which relies on the principle of scintillation or autoradiography.

In a further embodiment, the NMDA-receptor-associated disease or disorder is selected from the group consisting of neurodegenerative diseases or disorders, Alzheimer's disease, depressive disorders, Parkinson's disease, traumatic brain injury, stroke, migraine, alcohol withdrawal and chronic and neuropathic pain.

In a further aspect, the present invention is directed to a method for evaluating a putative NMDA-receptor antagonist comprising the steps:
(a) providing a compound for use in the present invention, wherein the compound comprises a tritium or ¹⁴C-atom,
(b) performing a step selected from the group consisting of:
   (i) measuring competitive binding affinity of a putative NMDA-receptor antagonist and the compound of step (a) in a liquid based-scintigraphy detection-based assay; and
   (ii) performing an in vitro or ex vivo autoradiography assay, wherein the putative NMDA-receptor antagonist is used for blocking or displacing the compound of step (a);
(c) determining whether the putative NMDA-receptor antagonist is an NMDA-receptor antagonist based on (i) the displacement of the compound of step (a) from the NMDA-receptor by the putative NMDA-receptor antagonist.

The putative NMDA-receptor antagonist can be any chemical molecule, including, e.g., small chemical entities and polypeptides, which is known to, or could be assumed to be an NMDA-receptor antagonist. The skilled person can assess any chemical molecule in the present method and determine whether this chemical molecule is an NMDA-receptor antagonist based on whether it displaces a compound for use in the invention or is displaced by a compound for use in the invention.

PET and SPECT imaging of NMDA receptors in the human body, in particular, the living human brain is a modern but already standard procedure in medical science and diagnosis. The average skilled person can routinely select an effective dosage, an effective formulation, the route and site of administration as well as all further parameters that are necessary to provide a meaningful PET or SPECT scan of the respective positron-emitting tracer compound in mammalian tissue. For an overview on PET imaging in general and PET imaging of NMDA-associated diseases and disorders in particular, reference is made to the articles of Sobrio et al., Mini-reviews in Medicinal Chemistry, 10, 870-886, 2010; Asselin et al., NeuroImage, 22, T131, 2004; Bressan et al., Biol Psychiatry, 58, 41-46, 2005; and Hartwig et al., Clin Pharmacol. Ther, 58, 165-178, 1995.

For diagnostic use the compounds of the invention may be administered in any conventional dosage form in any conventional manner. Routes of administration include oral, intravenous, intramuscular and subcutaneous injections. The preferred mode of administration is intravenous.

For liquid based-scintillation-based assays tritium and ¹⁴C-labeled compounds can be used in in vitro binding experiments, in vitro/ex vivo autoradiographies and ex vivo receptor occupancy experiments of GluN2B-antagonists in development. Advantages of tritium-labeled probes are the ease of radioactivity handling (less expenses for laboratory facility and higher safety compared to positron-emitters) and the high shelf-life of the product, which allows high throughput screening and several experiments at different time points with only one production batch. The quality of preclinical experiments is significantly improved as the commercially available tritiated radioligand currently used in GluN2B-targeted drug development is the non-selective [³H]ifenprodil. In contrast to [³H]ifenprodil, the compounds for use in the present invention are highly selective over sigma1 receptors and therefore provide an improved instrument to evaluate NMDA antagonists such as drug candidates.

The compounds may be administered alone or in combination with pharmaceutically acceptable excipients, e.g. excipients that enhance stability of the compounds, facilitate administration of pharmaceutical or diagnostic compositions containing them, provide increased dissolution or dispersion, diluents, buffers, viscosity-modifying agents, and the like, including other active ingredients. Advantageously such combination compositions utilize lower dosages of the conventional diagnostics, thus avoiding possible toxicity and adverse side effects incurred when those agents are used as mono-substances. The above-described compounds may be physically combined with conventional diagnostics or other excipients into a single pharmaceutical composition. Reference in this regard may be made to Cappola et al.: U.S. patent application Ser. No. 09/902,822, PCT/US 01/21860 und U.S. provisional application No. 60/313,527, each incorporated herein by reference in their entirety. Advantageously, the compounds of the invention may be administered alone or in combination with other biologically active compounds in a single or a multiple dosage form. The optimum percentage (w/w) of a compound of the invention in a dosage for PET/SPECT Scanning may vary and is within the purview of those skilled in the art. Alternatively, the compounds for PET/SPECT Scanning may be administered in several dosages.

As mentioned above, dosage forms of the compounds described herein include pharmaceutically acceptable excipients known to those of ordinary skill in the art. Methods for preparing such dosage forms are known (see, for example, H. C. Ansel and N. G. Popovish, Pharmaceutical Dosage Forms and Drug Delivery Systems, 5^{th} ed., Lea and Febiger (1990)). Dosage levels and requirements are well-recognized in the art and may be selected by those of ordinary skill in the art from available methods and techniques suitable for a particular patient. In some embodiments, dosage levels range from about 1-100 mg/dose for a 70 kg patient. Although one dose per PET/SPECT scan may be sufficient, up to 2 doses per PET/SPECT scan may be given. For intravenous doses, up to 2000 mg/PET or SPECT scan may be required. Reference in this regard is also made to U.S. provisional application No. 60/339,249. As the skilled artisan will appreciate, lower or higher dosages may be required depending on particular factors. For instance, specific doses and diagnostic procedures will depend on factors such as the patient's general health profile, the severity and course of the patient's disorder or disposition thereto, and the judgment of the diagnosing physician.

In the following, the invention will be illustrated by way of specific examples, none of which are to be interpreted as limiting the scope of the claims as appended.

### FIGURES

FIG. 1 shows a representative radiosynthesis of ¹⁸F-labeled demethylated benzazepine-1,7-diol derivatives.

FIG. 2 shows a representative radiosynthesis of [³H]Me-NB1, [³H]OF-Me-NB1, [³H]PF-NB1 and [³H]MF-Me-NB1.

FIG. 3 shows a representative radiosynthesis of [³H]OF-NB1 FIG. 4 shows a general methodology for the synthesis of benzazepin-1-ols.

FIG. 5 shows an autoradiography comparison between [¹⁸F]OF-NB1 and [¹⁸F]OF-Me-NB1 in rat and mouse brain.

FIG. 6 Shows an autoradiography comparison between [¹⁸F]PF-NB1 and [¹⁸F]PF-Me-NB1 in rat and mouse brain

FIG. 7 shows the selective and specific binding of [¹⁸F]OF-NB1 in vitro to GluN2B in rat and mouse brain.

FIG. 8 shows an ex vivo metabolite study of (R)-[¹⁸F]OF-NB1.

FIG. 9 shows a comparison of time-activity curves (TACS) between [¹⁸F]OF-NB1 and [¹⁸F]OFMe-NB1 in rat brain

FIG. 10 shows a comparison of time-activity curves (TACS) between [¹⁸F]PF-NB1 and [¹⁸F]PFMe-NB1 in rat brain.

FIG. 11 shows time-activity curves (TACS) of (R)-[¹⁸F]OF-NB1 using different doses of the experimental GluN2B antagonist CP-101,606 in rat brain.

### EXAMPLES

### Example 1—Chemistry

The general methodology for the synthesis of benzazepin-1-ols is known in the art, e.g. from Tewes et al., ChemMedChem 2010, 5, 687-695. A representative synthetic path as used for producing the labeled compounds for use in the present invention is shown in FIG. 4. The synthetic route of FIG. 4 can be adapted by commonly known methods to deliver derivatives of benzazepin-1-ols and substantially all of the compounds for use in the present invention. The skilled person will routinely adapt the synthetic route to be suitable for the synthesis of any PET ligand of the present invention.

### Example 2—F-18 Radiolabeling

[¹⁸F]fluoride was produced and trapped on an anion exchange cartridge (Waters SepPak Accell QMA cartridge carbonate, no pre-conditioning) and then eluted with a solution of Kryptofix 222 (6.3 mg/mL), K₂C₂O₄ (1 mg/mL) and K₂CO₃ (0.1 mg/mL) in MeCN/H₂O (4:1, 0.9 mL) followed by azeotropic drying with MeCN (3×1 mL) (Preshlock et al., ChemComm 2016). The reactivial was purged with air (20 mL) and the residue was re-dissolved in a solution of 6-8 mg boronic ester precursor 2a, 2b, 2c (FIG. 1) and 14 mg Cu(OTf)₂(py)₄ in 0.3 mL dry dimethylacetamide (DMA). The resulting solution was stirred at 120° C. for 20 minutes and subsequently diluted with 1.5 mL MeCN/H₂O (1:1). Upon addition of 0.4 mL aq. NaOH (10 M), the mixture was stirred at 95° C. for 15 min. The product was purified by semi-preparative HPLC (Agilent Eclipse XBD-C18 column, 250×9.4 mm, 5 m, 0.1% H₃PO₄ in H₂O (solvent A), MeCN (solvent B); 0.0-5.0 min, 20% B; 5.1-20.0 min, 20-35% B; 20.1-25.0 min, 35% B, 25.1-30.0 min, 35-60% B, 30.1-33.0 min, 60-90% B, 33.1-36.0 min, 90% B, 36.1-38.0 min, 90-20% B, 38.1-42.0 min, 20% B, flow rate 4 mL/min, UV detection at 230 nm). Collected fractions were diluted with 35 mL water and the product was trapped on a C18 light cartridge (Waters, pre-conditioned with 5 mL EtOH and 5 mL water). The cartridge was washed with 5 mL of water, followed by product elution with 0.5 mL EtOH. The product was formulated with 5% EtOH in saline to afford either [¹⁸F]OF-Me-NB1 3a, [¹⁸F]PF-Me-NB1 3b or [¹⁸F]MF-Me-NB1 3c (FIG. 1). Alternatively, the ethanol was evaporated at 90° C. followed by azeotropic drying with MeCN (3×0.8 mL). The residue was cooled down in an ice bath before re-dissolving it in 0.4 mL DCM. The vial was kept in the ice bath as 0.4 ml of BBr₃ (1 M in DCM) was added simultaneously, followed by stirring at rt for 15 min. The DCM was evaporated to dryness and subsequently re-dissolved in aq. 0.1% H₃PO₄/MeCN (5:1, 3 mL). Afterwards, the product was purified by semi-preparative HPLC (Agilent Eclipse XBD-C18 column, 250×9.4 mm, 5 m, 0.1% H₃PO₄ in H₂O (solvent A), MeCN (solvent B); 0.0-5.0 min, 20% B; 5.1-20.0 min, 20-35% B; 20.1-25.0 min, 35% B, 25.1-30.0 min, 35-60% B, 30.1-33.0 min, 60-90% B, 33.1-36.0 min, 90% B, 36.1-38.0 min, 90-20% B, 38.1-42.0 min, 20% B, flow rate 4 mL/min, UV detection at 230 nm). Collected fractions were diluted with 35 mL water and the product was trapped on a C18 light cartridge (Waters, pre-conditioned with 5 mL EtOH and 5 mL water). The cartridge was washed with 5 mL of water, followed by product elution with 0.5 mL EtOH. The product was formulated with 5% EtOH to afford either [¹⁸F]OF-NB1 4a, [¹⁸F]PF-NB1 4b or [¹⁸F]MF-NB1 4c (FIG. 1). Quality control was performed using an analytical HPLC system (Waters Atlantis T3 column, 150×4.6 mm, 3 m, 0.1% TFA in H₂O (solvent A), MeCN (solvent B); 0.0-10.0 min, 40-60% B; 10.1-11.0 min, 60-40% B; 11.1-15.0 min, 40% B, flow rate 1 mL/min, UV detection at 230 nm). Identities of the radioligands were confirmed by co-injection with the corresponding nonradioactive reference compounds and molar activities were calculated based on established UV calibration curves of the reference compounds. The radiochemical conversion (RCC) ranged between 9-25% and radiochemical purities >95%. Molar activities ranged from 61-168 GBq/mol at the end of synthesis.

### Example 3—Autoradiography

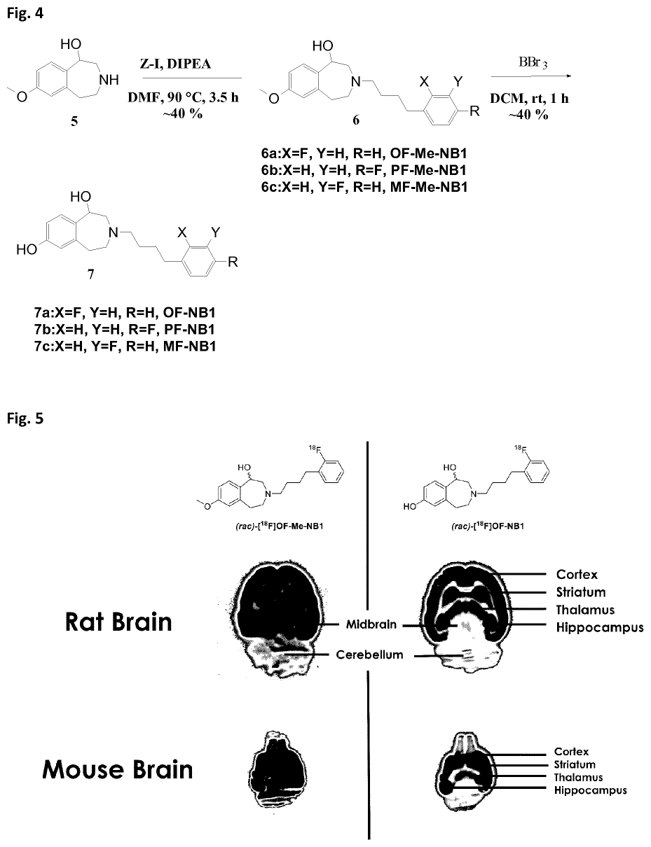

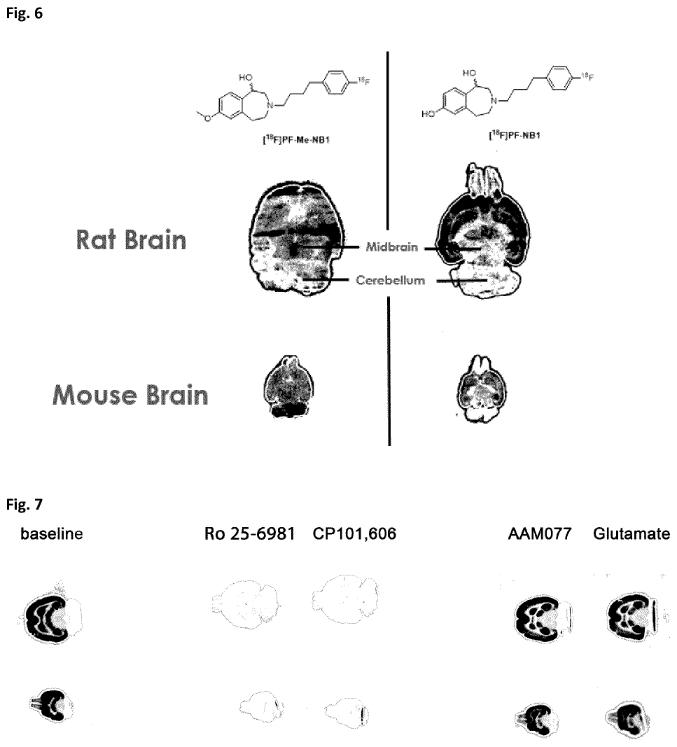

Rodent brain tissue was embedded in Tissue-Tek® (O.C.T.™ Tissue-Tek®, Sakura Finetek Europe B.V., Alphen aan den Rijn, Netherlands). Horizontal rat and mouse brain sections of 10 m thickness were prepared on a cryostat (Cryo-Star HM 560 MV; Microm, Thermo Scientific, Wilmington, Del., USA). The tissue sections were mounted to SuperFrost Plus slides (Menzel, Braunschweig, Germany) and stored at −20° C. until further use. Prior to the autoradiography experiments, brain slices were initially thawed for 15 min on ice and subsequently preconditioned for 10 min at 0° C. in a buffer (pH 7.4) containing 30 mM HEPES, 0.56 mM MgCl₂, 110 mM NaCl, 3.3 mM CaCl₂, 5 mM KCl and 0.1% fatty acid free bovine serum albumin (BSA). Upon drying, the tissue sections were incubated with 1 mL of the respective radioligand (3 nM) for 15 minutes at 21° C. in a humidified chamber. For σ1R-blockade, 1 μM solution of either SA4503, fluspidine or (+)pentazocine was added to the radiotracer solution. For GluN2B-blockade experiments, 1 μM solution of either CERC-301, EVT 101 or CP101,606 was added to the radiotracer solution. The brain slices were washed for 5 min with a buffer (pH 7.4) containing 30 mM HEPES, 0.56 mM MgCl₂, 110 mM NaCl, 3.3 mM CaCl₂, 5 mM KCl and 0.1% fatty acid free bovine serum albumin (BSA) and further washed twice 3 min in a second buffer with the same ionic composition but without BSA. Tissue sections were dipped twice in distilled water, subsequently dried and exposed to a phosphor imager plate (Fuji, Dielsdorf, Switzerland) for 30 minutes. The films were scanned in a BAS5000 reader (Fuji) and images were generated using AIDA 4.50.010 software (Raytest Isotopenmessgeräte GmbH, Straubenhardt, Germany). Typical autoradiographies of methylated and demethylated NB1 are shown in FIGS. 5, 6 and 7. The distribution of the tracers conforms to the expression distribution of the GluN2B over the brain. Additionally, to establish specificity, GluN2B blockers (Ro 25-6981, CP101.606 (Sigma-Aldrich, Buchs, Switzerland)) were used. As a negative control for establishing selectivity, GluN2A blocker NVP-AAM077 (Sigma-Aldrich, Buchs, Switzerland) and the endogenous ligand glutamate were used.

### Example 4—PET Scans of Rat Brain and Time-Activity Curves and Receptor Occupancy

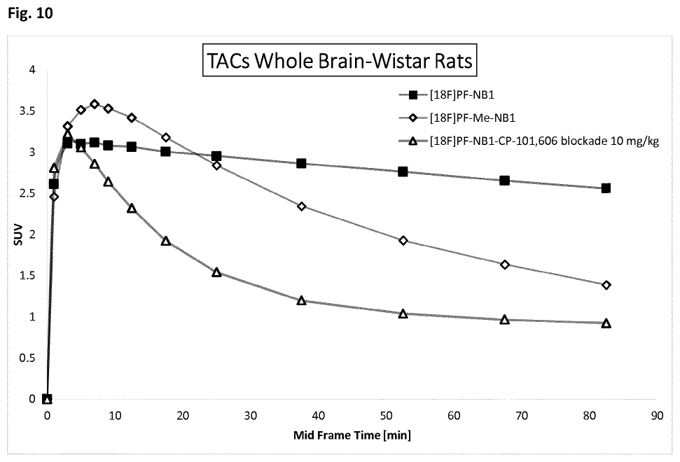

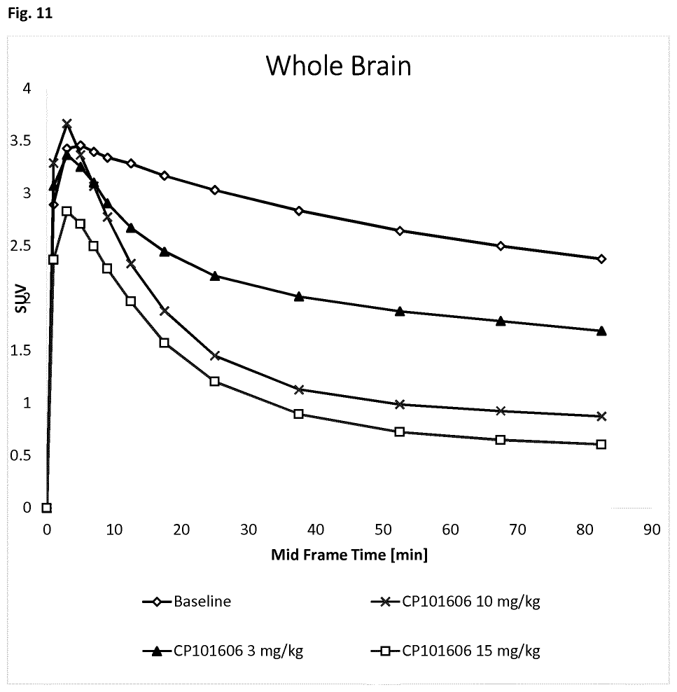

Wistar rats were anesthetized with isoflurane and scanned for a period of 90 min in a PET/CT scanner (Super Argus, Sedecal, Madrid, Spain) upon tail-vein injection of 15-38 MBq, 0.6-1.7 nmol/kg (rats) of methylated and demethylated radiolabelled ligands. For anatomical orientation, PET scans were followed by computed tomography. Dose-response and receptor occupancy in Wistar rats were conducted by tail-vein injection of different doses (3, 10 and 15 mg/kg) of CP101,606 (GluN2B-antagonist, Sigma-Aldrich, Buchs, Switzerland) shortly before tracer administration The obtained data was reconstructed in user-defined time frames with a voxel size of 0.3875×0.3875×0.775 mm³ as previously described by our group (Haider et al., Eur. J. Med. Chem. 2018). Time-activity curves (TACs) were deducted by PMOD v3.7 (PMOD Technologies, Zurich, Switzerland) with predefined regions of interest. The results are given as standardized uptake values (SUVs), indicating the decay-corrected radioactivity per cm³ divided by the injected dose per gram body weight. Receptor occupancy evaluations were carried out as previously reported (Haider et al., Eur. J. Med. Chem. 2018). Baseline TACs for the methylated and demethylated benzazepines are depicted in FIGS. 9 and 10. TACs resulting from a typical receptor occupancy study using (R)[¹⁸F]-OF-NB1 is shown in FIG. 11.

### Example 5—In Vitro Binding Affinities

The conduction of the binding competition assay for the GluN2B is already known from the current state of the art (Szermerski et al, ChemMedChem, 2018) as well as the assay for 61 receptor (σ1R) (Chu et al, Current Protocols in Pharmacology, 2015). Portions of rat membrane homogenates of ˜1 mg protein/mL (for GluN2B subunit IC₅₀ determination) and ˜2 mg protein/mL (for σ1R IC₅₀ determination) were used in the assay (determined by the method of Bradford). The radioligands used were [³H]ifenprodil (Perkin Elmer) for the GluN2B binding competition assay and [³H](+)-pentazocine (Perkin Elmer) for the σ1R binding competition assay. For the tested ligands, a dilution series of 8 different concentrations were prepared ranging from 30 pM up to 30 nM. For the GluN2B subunit binding assay, [³H]ifenprodil was incubated together with the substrate and 1 mg/ml total protein in HEPES buffer (30 mM, 110 mM NaCl, 5 mM KCl, 2.5 mM CaCl₂), 1.2 mM MgCl2, pH 7.4) in a total volume of 200 μl at 25° C. for 60 min under mechanical shaking (110 Rpm). The σ1R binding competition binding assay was performed similarly with modifications in the total protein concentration, incubation time and temperature of 2 mg protein/mL, 37° C. and 150 min, respectively. Termination of the incubation was completed by dilution with 3 ml HEPES buffer followed by filtration through glass microfiber filters (Whatman GF/C 25 mm) pre-incubated in 0.05% polyethylenimine solution. To measure the activity, scintillation fluid (10 ml/scintillation vial, Ultima Gold, Perkin Elmer) was utilized and the activity was measured by a Packard 2200CA TRI-CARB liquid scintillation analyzer. The resulting IC₅₀ values were transformed into Ki values using the equation of Cheng and Prusoff (Cheng Y, Prusoff WH, 1973, Biochem Pharmacol. 22 (23)). The Ki values for methylated (OF-Me-NB1 and PF-Me-NB1) and demethylated (OF-NB1 and PF-NB1) are depicted in Table 1 below.

TABLE 1
GluN2B subunit and σ1R Ki values
of the benzoadepine-1-ol derivatives

| | GluN2B Affinity | σ1R Affinity | Selectivity |
|---|---|---|---|
| Compounds | in nM | in nM | (σ1R/GluN2B) |
| OF-Me-NB1 | 37 | 32 | 0.9 |
| PF-Me-NB1 | 56 | 12 | 0.2 |
| OF-NB1 | 10.36 ± 4.75 (n = 3) | 410 | 39.6 |
| PF-NB1 | 10.44 ± 3.90 (n = 3) | 544 | 52.1 |

### Example 6—In Vitro Binding Affinities

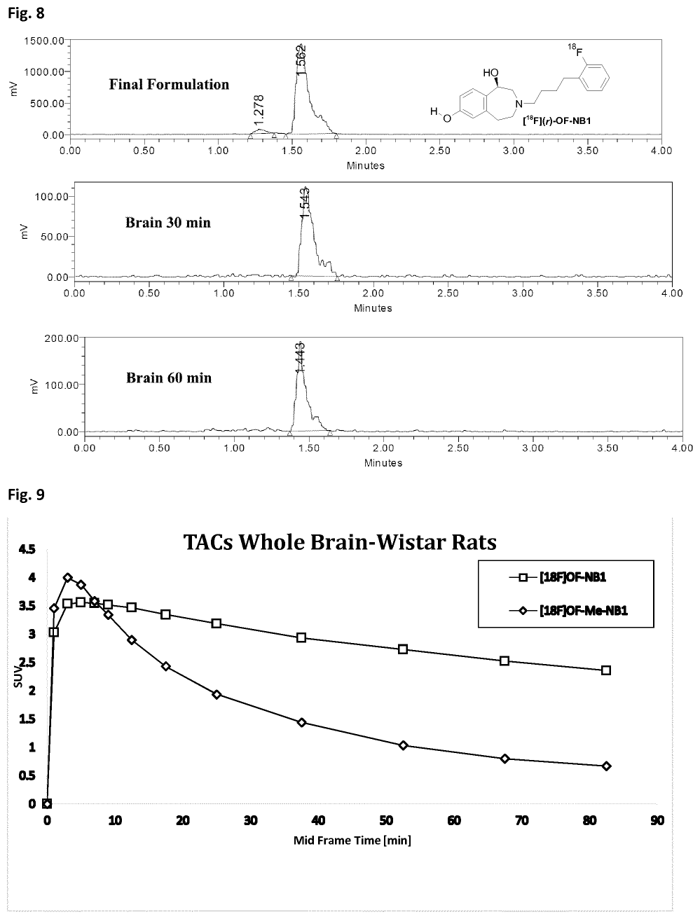

Wistar rats were injected with 242-704 MBq (14.8-27.5 nmol/kg) of (R)-[¹⁸F]OF-Me-NB1 and (R)-[¹⁸F]OF-NB1. Samples of the brain extracts at predefined times (15, 30 and 60 min) were obtained and analyzed by radio-UPLC as previously reported (Haider et al., Eur. J. Med. Chem. 2018). This class of compounds was found to be metabolically stable in the brain as no radiometabolites were detected up to 60 min in the brain. A typical metabolite study is shown in FIG. 8 for (R)-[¹⁸F]OF-NB1.

### Example 7—Tritium Labeling

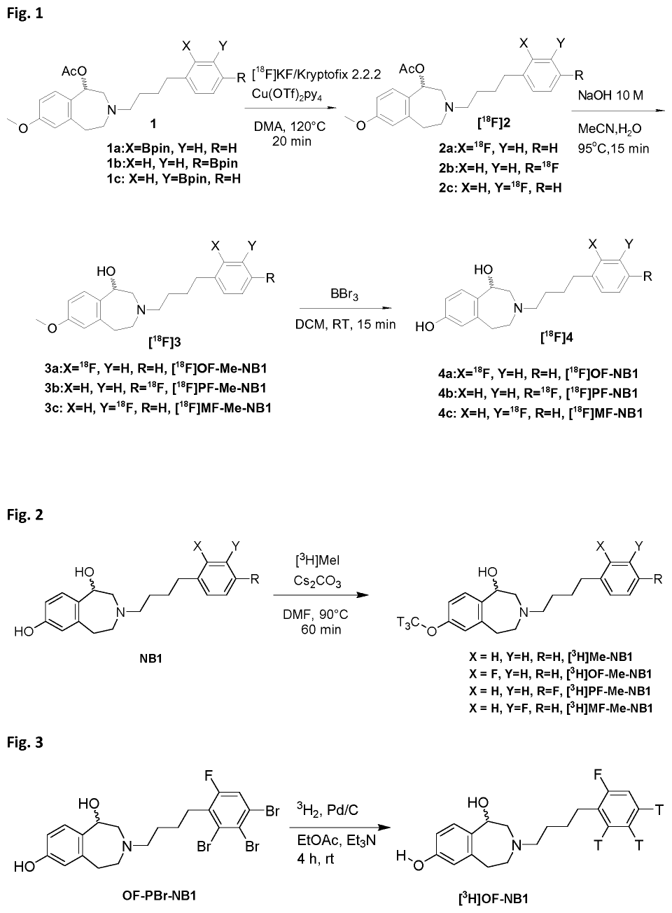

For methylated ligands, a typical tritium-labeling approach is presented in FIG. 2. To a stirred solution of (R)-NB1 (0.5 mg, 1.6 μmol) in dry DMF (0.2 mL) was added cesium carbonate (2.5 mg, 7.7 μmol) and the resulting suspension was stirred for 5 min at ambient temperature. A solution of [³H]iodomethane (15 MBq, ARC, St Lous, US, molar activity 1.4 GBq/mol) in DMF (0.1 mL) was added dropwise and the reaction mixture was stirred for 1 h at 90° C. The mixture was diluted with aqueous TFA buffer (0.1%, 0.7 mL) and subsequently injected into a semiproparative HPLC system (Merck Hitachi) equipped with L-6200A pump system, a D-6000 interface, an L-4250 UV-VIS detector and a semipreparative column (Waters Sunfire Prep C18 column (150×10.0 mm, 5 m). A gradient system of 0.1% aqueous TFA (solvent A) and MeCN (solvent B) was used for the purification as follows: 0-5 min 20% B, 5-40 min 20-95% B, 40-41 min 95-20% B. The product was collected, diluted with 4 mL of water and passed through a Waters Sep Pak C18 light cartridge (preconditioned with 5 mL ethanol and 5 mL water). The cartridge was washed with 5 mL of water and the product eluted with 1 mL of ethanol. Quality control was performed with the same HPLC system and gradient. The synthesis was successfully performed under carrier-added and non-carrier added conditions and the final product was stored at −20° C. Another example of introducing tritium into the demethylated-NB1 ligands would be through aromatic tritiation as shown in FIG. 3 (McCarthy et al, J. Label. Compd. Radiopharm, 1997).

1.-19. (canceled)

20. A method for the diagnosis of NMDA receptor-associated diseases or disorders, the method comprising:
(a) administering to a patient or a sample of a patient in need of such diagnosis a compound of formula (I) in an amount effective for PET imaging, SPECT imaging, liquid based-scintillation- and/or autoradiography-based assays of NMDA receptors,
(b) recording at least one PET scan, SPECT scan, liquid based-scintillation or autoradiography result, optionally by ex vivo analysis, and
(c) diagnosing an NMDA-receptor-associated disease or disorder from an abnormal NMDA receptor expression pattern on the PET scan, the SPECT scan or the autoradiography result, or an abnormal NMDA receptor expression pattern in the liquid-based scintillation result, wherein
the chemical structure of formula (I) is: 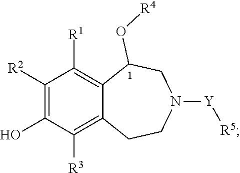
at least one atom of formula (I) is a radiolabeled atom suitable for detection in a method selected from the group consisting of positron emission tomography (PET), single-photon emission computed tomography (SPECT), liquid based-scintillation counting assays, and autoradiography;
one of R¹, R² and R³ is independently selected from the group consisting of hydrogen, deuterium, tritium, fluorine, chlorine, bromine, iodine, —OH, —CN, —NO₂, —NH₂, —SH₂, —(C₁-C₄)alkyl, fluorinated —(C₁-C₄)alkyl, and fluorinated —O(C₁-C₄)alkyl, and the other of R¹ to R³ are hydrogen or fluorine; wherein T is ³H;
R⁴ is selected from the group consisting of hydrogen, —(C₁-C₄)alkyl, fluorinated —(C₁-C₄)alkyl, chlorinated —(C₁-C₄)alkyl, brominated —(C₁-C₄)alkyl, deuterated —(C₁-C₄)alkyl, and tritiated —(C₁-C₄)alkyl;
Y is selected from the group consisting of (C₁-C₆)alkyl, (C₁-C₆)alkyl substituted with at least one deuterium, tritium, ¹⁸F, ¹⁹F, chlorine, bromine, iodine, OH, or a combination thereof, (C₁-C₆)alkoxyalkyl, (C₁-C₆)polyethyleneglycoyl, and (C₁-C₆)heteroalkyl;
R⁵ is selected from the group consisting of substituted or non-substituted (C₅-C₆)aryl, and substituted or non-substituted (C₅-C₆)heteroaryl; and
YR⁵ is selected from the group consisting of: 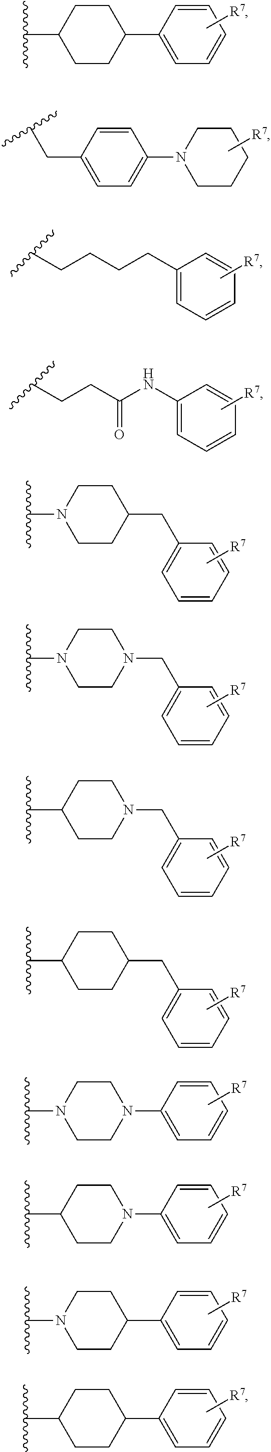
wherein R⁷ is one or more hydrogen, deuterium, tritium, fluorine, chlorine, bromine, iodine or (C₁-C₇)alkyl substituted with at least one deuterium, tritium, fluorine, chlorine, bromine, iodine or OH;
and pharmaceutically acceptable salts or solvates thereof.

21. The method of claim 1, wherein the radiolabeled atom is selected from the group consisting of ³H-atom, ¹¹C-atom, ¹⁴C-atom, ¹⁸F-atom, ¹³N-atom, ¹⁵O-atom, ¹²³I-atom, ¹²⁴I-atom, ¹²⁵I-atom, and ¹³¹I-atom.

22. The method of claim 1, wherein the radiolabeled atom is a positron emitting radiometal.

23. The method of claim 22, wherein the positron emitting radiometal is ⁶⁴Cu or ⁶⁸Ga.

24. The method of claim 21, wherein the radiolabeled atom is a ¹¹C-atom, ¹⁸F-atom, ¹²³I-atom, ¹²⁴I-atom, ¹²⁵I-atom, or ¹³¹I-atom.

25. The method of claim 20, wherein at least one of R⁴ or R⁵ comprises a ¹¹C-atom, ¹⁸F-atom, ¹²³I-atom, ¹²⁴I-atom, ¹²⁵I-atom, or ¹³¹I-atom.

26. The method of claim 25, wherein R⁴ is —OCH₂CH₂—¹⁸F, —OCH₂CH₂CH₂—¹⁸F, OCH₂CH₂—¹²³I, or —OCH₂CH₂CH₂—¹²³I, R⁶ is —¹⁸F or —¹²³I, or a combination thereof.

27. The method of claim 20, wherein one of R¹, R² and R³ is independently selected from the group consisting of —CH₂F, —CD₂F, —CT₂F, FCH₂CH₂—, FCH₂CH₂CH₂—, OCH₂F, —OCD₂F, FCH₂CH₂O—, and FCH₂CH₂CH₂O—.

28. The method of claim 20, wherein R⁴ is selected from the group consisting of —CH₂F, —CD₂F, —CT₂F, FCH₂CH₂—, FCH₂CH₂CH₂—, FCD₂CD₂-, FCD₂CD₂CD₂-, FCT₂CT₂-, FCT₂CT₂CT₂-, —CH₂Cl, —CD₂Cl, —CT₂I, ClCH₂CH₂—, ClCH₂CH₂CH₂—, ClCD₂CD₂-, ClCD₂CD₂CD₂-, ClCT₂CT₂-, ClCT₂CT₂CT₂-, —CH₂I, —CD₂I, —CT₂I, ICH₂CH₂—, ICH₂CH₂CH₂—, ICD₂CD₂-, ICD₂CD₂CD₂-, ICT₂CT₂-, and ICT₂CT₂CT₂-.

29. The method of claim 20 or 28, wherein R¹ to R³ are hydrogen, fluorine, chlorine, bromine or iodine.

30. The method of claim 20, wherein Y is selected from the group consisting of C₅-alkyl, C₄-alkyl, CH₂—CH₂—FCH—CH₂N—, CH₂—FCH—CH₂CH₂N—, CH₂CH₂—ICH—CH₂N—, CH₂—ICH—CH₂CH₂N—, CH₂CH₂—CD₂-CH₂N—, CH₂—CD₂-CD₂CH₂N—, CH₂CH₂—CT₂-CH₂N—, CH₂—CT₂-CT₂CH₂N—, or CH₂—CHOH—CH₂—CH₂N—, —(CH₂)₂—O—(CH₂)₂—R⁵, —(CH₂)₃—O—R⁵, —(CH₂)₄—O—R⁵, —(CH₂)₃—X—R⁵ and —(CH₂)₄—X—R⁵, wherein X is sulfur or SO₂, 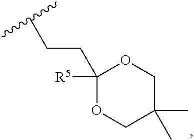
—(CH₂)₃—CO—R⁵, —(CH₂)₂—CO—N(CH₃)—CH₂—R⁵, and —CO—(CH₂)₃—R⁵.

31. The method of claim 30, wherein R⁵ is selected from the group consisting of substituted or non-substituted phenyl or pyridyl.

32. The method of claim 20, wherein R⁵ is selected from the group consisting of substituted or non-substituted phenyl or pyridyl.

33. The method of claim 20, wherein one of R¹, R² and R³ is independently selected from the group consisting of —H, -D, -T, —CH₃, —CH₂F, —CD₂F, FCH₂CH₂—, FCH₂CH₂CH₂—, —OCH₃, —OCH₂F, —OCD₂F, FCH₂CH₂O— and FCH₂CH₂CH₂O—, and the other of R¹ to R⁴ are hydrogen or fluorine.

34. The method of claim 20, wherein R¹ is selected from the group consisting of —CH₃, —CH₂F, —CD₂F, FCH₂CH₂—, FCH₂CH₂CH₂—, —OCH₃, —OCH₂F, —OCD₂F, FCH₂CH₂O— and FCH₂CH₂CH₂O—, and R², R³ and R⁴ are hydrogen or fluorine.

35. The method of claim 20, wherein R⁴ is selected from the group consisting of hydrogen, —CH₂F, —CD₂F, FCH₂CH₂—, and FCH₂CH₂CH₂—.

36. The method of claim 20, wherein Y is selected from the group consisting of —(CH₂)ᵢ—R⁵ and —(CH₂)ₑ—O—(CH₂)_{f}—R⁵, wherein i is an integer from 2 to 6, and e and f are independently selected from 1, 2 or 3.

37. The method of claim 20, wherein R⁵ is selected from the group consisting of phenyl, 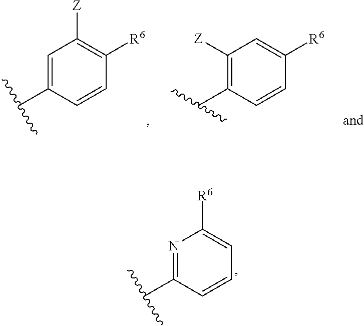
wherein Z is selected from the group consisting of hydrogen, deuterium, tritium, fluorine, chlorine, bromine, iodine, cyano and nitrile, and wherein R⁶ is selected from the group consisting of hydrogen, deuterium, tritium, fluorine, chlorine, bromine, iodine, cyano, —CH₂F, —CD₂F, FCH₂CH₂—, FCH₂CH₂—, and FCH₂CH₂CH₂—.

38. The method of claim 37, wherein R⁶ is selected from the group consisting of hydrogen, tritium, fluorine and iodine.

39. The method of claim 20, wherein R⁶ is selected from the group consisting of hydrogen, tritium, fluorine and iodine.

40. The method of claim 20, wherein the compound is R-configured at carbon 1.

41. The method of claim 20, wherein the compound is S-configured at carbon 1.

42. The method of claim 20, wherein
R¹ is selected from the group consisting of —OCH₃, —OCH₂F, —OCD₂F, FCH₂CH₂O— and FCH₂CH₂CH₂O— and R², R³ and R⁴ are hydrogen,
R⁴ is hydrogen,
Y is —(CH₂)₄—, and
R⁵ is substituted or unsubstituted phenyl.

43. The method of claim 20, wherein
R⁴ is selected from the group consisting of hydrogen, —CH₂F, —CD₂F, FCH₂CH₂—, and FCH₂CH₂CH₂—,
Y is —(CH₂)₄— or —(CH₂)₂—O—(CH₂)₂—, and
R⁵ is selected from the group consisting of phenyl, 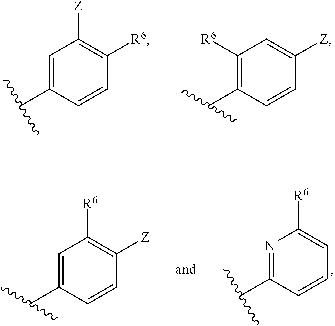
wherein Z is hydrogen, tritium or nitrile and wherein R⁶ is selected from the group consisting of fluorine, iodine, —CH₂F, —CD₂F, FCH₂—, FCH₂CH₂—, and FCH₂CH₂CH₂—.

44. The method of claim 20, wherein
R⁴ is hydrogen,
Y is (CH₂)₄, and
R⁵ is substituted or un-substituted phenyl.

45. The method of claim 20, wherein the compound is selected from the group consisting of 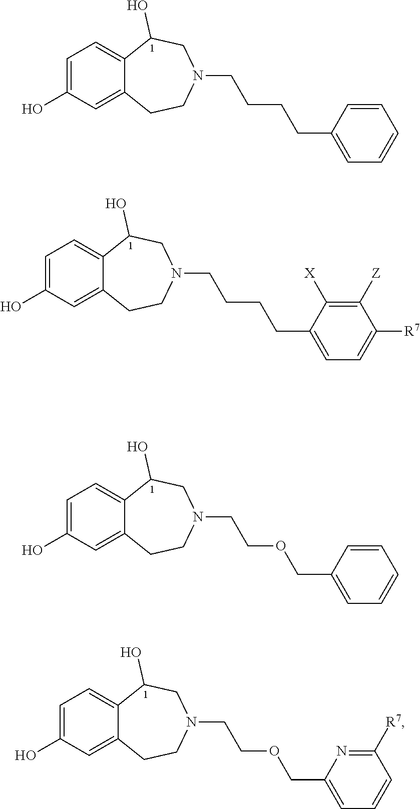
wherein
R⁷ is selected from the group consisting of fluorine, chlorine, bromine, iodine, deuterium, tritium, —CH₂F, —CD₂F, FCH₂CH₂—, FCH₂CH₂—, and FCH₂CH₂CH₂—; and
X and Z are independently selected from the group consisting of hydrogen, deuterium, tritium, fluorine, iodine and nitrile.

46. The method of claim 20, wherein the compound is selected from the group consisting of 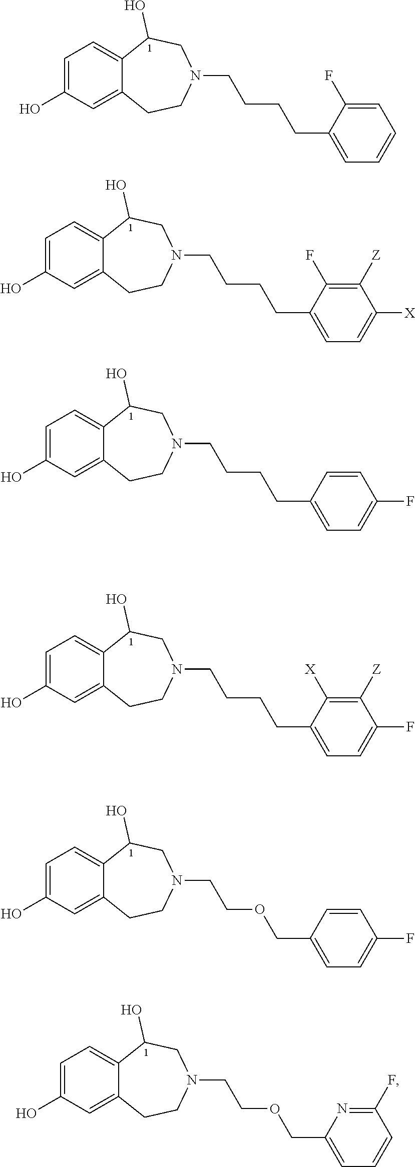
wherein
X and Z are independently selected from the group consisting of hydrogen, deuterium, tritium, fluorine, iodine and nitrile; and
the fluorine is ¹⁹F if X and/or Z are tritium and the fluorine is ¹⁸F if X and/or Z are not.

47. The method of claim 20, wherein the NMDA-receptor-associated disease or disorder is selected from the group consisting of neurodegenerative diseases or disorders, Alzheimer's disease, depressive disorders, Parkinson's disease, traumatic brain injury, stroke, migraine, alcohol withdrawal and chronic and neuropathic pain.

48. A method for evaluating a putative NMDA-receptor antagonist, the method comprising:
(a) providing a compound of formula (I),
(b) performing a step selected from the group consisting of:
(i) measuring competitive binding affinity of a putative NMDA-receptor antagonist and the compound of step (a) in a liquid scintigraphy detection-based assay; and
(ii) performing an in vitro or ex vivo autoradiography assay, wherein the putative NMDA-receptor antagonist is used for blocking or displacing the compound of step (a);
(c) determining whether the putative NMDA-receptor antagonist is an NMDA-receptor antagonist based on the displacement of the compound of step (a) from the NMDA-receptor by the putative NMDA-receptor antagonist,
wherein
the compound comprises a tritium or ¹⁴C-atom;
the chemical structure of formula (I) is: 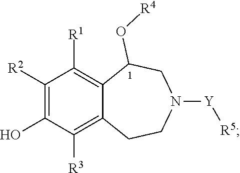
at least one atom of formula (I) is a radiolabeled atom suitable for detection in a method selected from the group consisting of positron emission tomography (PET), single-photon emission computed tomography (SPECT), liquid based-scintillation counting assays, and autoradiography;
one of R¹, R² and R³ is independently selected from the group consisting of hydrogen, deuterium, tritium, fluorine, chlorine, bromine, iodine, —OH, —CN, —NO₂, —NH₂, —SH₂, —(C₁-C₄)alkyl, fluorinated —(C₁-C₄)alkyl, and fluorinated —O(C₁-C₄)alkyl, and the other of R¹ to R³ are hydrogen or fluorine; wherein T is ³H;
R⁴ is selected from the group consisting of hydrogen, —(C₁-C₄)alkyl, fluorinated —(C₁-C₄)alkyl, chlorinated —(C₁-C₄)alkyl, brominated —(C₁-C₄)alkyl, deuterated —(C₁-C₄)alkyl, and tritiated —(C₁-C₄)alkyl;
Y is selected from the group consisting of (C₁-C₆)alkyl, (C₁-C₆)alkyl substituted with at least one deuterium, tritium, ¹⁸F, ¹⁹F, chlorine, bromine, iodine, OH, or a combination thereof, (C₁-C₆)alkoxyalkyl, (C₁-C₆)polyethyleneglycoyl, and (C₁-C₆)heteroalkyl;
R⁵ is selected from the group consisting of substituted or non-substituted (C₅-C₆)aryl, and substituted or non-substituted (C₅-C₆)heteroaryl; and
YR⁵ is selected from the group consisting of: 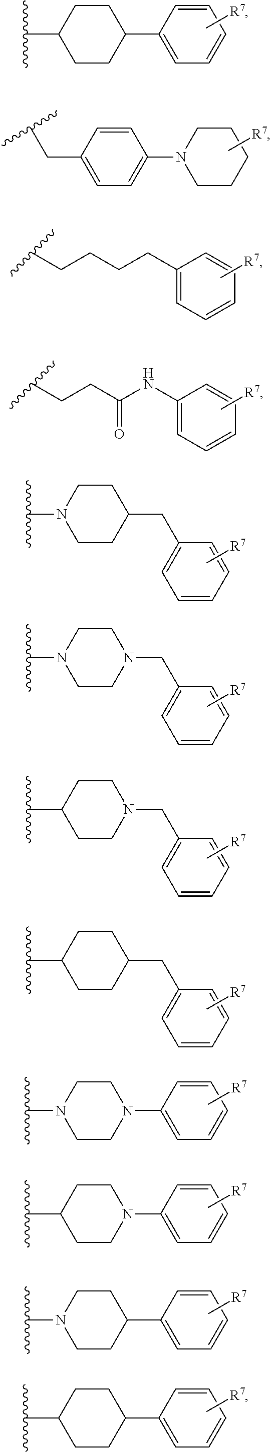
wherein R⁷ is one or more hydrogen, deuterium, tritium, fluorine, chlorine, bromine, iodine or (C₁-C₇)alkyl substituted with at least one deuterium, tritium, fluorine, chlorine, bromine, iodine or OH;
and pharmaceutically acceptable salts or solvates thereof.
